(19) United States
(12) Patent Application Publication
(10) Pub. No.: US 2018/0021569 A1
(43) Pub. Date: Jan. 25, 2018

(54) SYSTEMS AND METHODS FOR MAKING AND USING AN ELECTRICAL STIMULATION SYSTEM FOR STIMULATION OF DORSAL ROOT GANGLIA

(71) Applicant: Boston Scientific Neuromodulation Corporation, Valencia, CA (US)

(72) Inventor: Anne Margaret Pianca, Santa Monica, CA (US)

(21) Appl. No.: 15/656,698

(22) Filed: Jul. 21, 2017

Related U.S. Application Data
(60) Provisional application No. 62/366,454, filed on Jul. 25, 2016.

(51) Int. Cl. A61N 1/05; A61M 25/09; A61B 17/34; A61N 1/36

(52) U.S. Cl. CPC A61N 1/0551; A61N 1/0558; A61N 1/36062; A61M 25/09; A61B 17/3417; A61B 2017/3456

(57) ABSTRACT

A method for implanting a lead for stimulation of a dorsal root ganglion of a patient includes advancing a distal portion of a guidewire using an introducer into an epidural space of the patient and through a foramen of the patient to a position near the dorsal root ganglion, the guidewire including an electrode in the distal portion of the guidewire; mapping a region around the dorsal root ganglion using the electrode of the guidewire to identify a lead implantation site; removing the introducer; and advancing the lead over the guidewire, with a portion of the guidewire disposed in a lumen of the lead, to position a distal portion of the lead at the lead implantation site.

### CROSS-REFERENCE TO RELATED APPLICATIONS

This application claims the benefit under 35 U.S.C. §119(e) of U.S. Provisional Patent Application Ser. No. 62/366,454, filed Jul. 25, 2016, which is incorporated herein by reference.

### FIELD

The present invention is directed to the area of implantable electrical stimulation systems and methods of making and using the systems. The present invention is also directed to electrical stimulation systems for stimulation of dorsal root ganglia, as well as methods of making and using the electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Sacral nerve stimulation has been used to treat incontinence, as well as a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

Dorsal root ganglia are nodules of cell bodies disposed along the dorsal roots of spinal nerves. Dorsal root ganglia are disposed external to the epidural space. Dorsal root ganglia, however, are disposed in proximity to the spinal cord and the vertebral column.

### BRIEF SUMMARY

One embodiment is a method for implanting a lead for stimulation of a dorsal root ganglion of a patient. The method includes advancing a distal portion of a guidewire using an introducer into an epidural space of the patient and through a foramen of the patient to a position near the dorsal root ganglion, the guidewire including an electrode in the distal portion of the guidewire; mapping a region around the dorsal root ganglion using the electrode of the guidewire to identify a lead implantation site; removing the introducer; and advancing the lead over the guidewire, with a portion of the guidewire disposed in a lumen of the lead, to position a distal portion of the lead at the lead implantation site.

In at least some embodiments, advancing the distal portion of the guidewire includes advancing the introducer and the distal portion of the guidewire through the foramen of the patient. In at least some embodiments, the introducer has a flat, blunt tip to facilitate penetration of scar tissue around the foramen. In at least some embodiments, mapping the region around the dorsal root ganglion includes stimulation of patient tissue using the electrode of the guidewire. In at least some embodiments, mapping the region around the dorsal root ganglion includes receiving electrical signals from patient tissue using the electrode of the guidewire.

In at least some embodiments, the introducer is no more than 20 gauge. In at least some embodiments, the method further includes repositioning the distal portion of the guidewire to another site relative to the dorsal root ganglion. In at least some embodiments, the introducer includes a reinforced mesh to reduce kinking.

Another embodiment is a method for implanting a lead for stimulation of a dorsal root ganglion of a patient. The method includes advancing a distal portion of a guidewire through an epidural space of the patient and through a foramen of the patient to a position near the dorsal root ganglion, the guidewire including an electrode in the distal portion of the guidewire; mapping a portion of the patient tissue adjacent the distal portion of the guidewire using the electrode; repositioning the distal portion of the guidewire to a lead implantation site relative to the dorsal root ganglion and mapping an additional portion of the patient tissue using the electrode; and advancing the lead over the guidewire, with a portion of the guidewire disposed in a lumen of the lead, to position a distal portion of the lead at the lead implantation site.

In at least some embodiments, advancing the distal portion of the guidewire includes advancing the guidewire through an introducer. In at least some embodiments, advancing the distal portion of the guidewire further includes advancing the introducer and the distal portion of the guidewire through the foramen of the patient. In at least some embodiments, the introducer has a flat, blunt tip to facilitate penetration of scar tissue around the foramen. In at least some embodiments, the introducer is no more than 20 gauge.

In at least some embodiments, mapping the portion of the patient tissue includes stimulating patient tissue using the electrode of the guidewire. In at least some embodiments, mapping the portion of the patient tissue includes receiving electrical signals from patient tissue using the electrode of the guidewire. In at least some embodiments, the introducer includes a reinforced mesh to reduce kinking.

Yet another embodiment is a kit for implanting a lead for stimulation of a dorsal root ganglion of a patient. The kit includes a guidewire with an electrode disposed at a distal end of the guidewire; an introducer having a lumen for receiving the guidewire; and a lead having a lead body and electrodes disposed along a distal end of the lead body, the lead body defining a central lumen for receiving the guidewire.

In at least some embodiments, the introducer has a blunt tip for penetrating scar tissue. In at least some embodiments, the introducer is no more than 20 gauge. In at least some embodiments, the introducer includes a reinforced mesh to reduce kinking.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic view of another embodiment of an electrical stimulation system that includes a percutaneous lead body coupled to a control module, according to the invention;

FIG. 1B is a schematic perspective view of the distal portion of another embodiment of a lead with segmented electrodes, according to the invention;

FIG. 2A is a schematic view of one embodiment of a plurality of connector assemblies disposed in the control module of FIG. 1A, the connector assemblies configured and arranged to receive the proximal portions of the lead bodies of FIG. 1A, according to the invention;

FIG. 2B is a schematic view of one embodiment of a proximal portion of the lead body of FIG. 1, a lead extension, and the control module of FIG. 1A, the lead extension configured and arranged to couple the lead body to the control module, according to the invention;

FIG. 3A is a schematic transverse cross-sectional view of spinal nerves extending from a spinal cord, the spinal nerves including dorsal root ganglia;

FIG. 3B is a schematic perspective view of a portion of the spinal cord of FIG. 3A disposed in a portion of a vertebral column with the dorsal root ganglia of FIG. 3A extending outward from the vertebral column;

FIG. 3C is a schematic top view of a portion of the spinal cord of FIG. 3A disposed in a vertebral foramen defined in a vertebra of the vertebral column of FIG. 3B, the vertebra also defining intervertebral foramina extending between an outer surface of the vertebra and the vertebral foramen, the intervertebral foramina providing an opening through which the dorsal root ganglia of FIG. 3B can extend outward from the spinal cord of FIG. 3B;

FIG. 3D is a schematic side view of two vertebrae of the vertebral column of FIG. 3B, the vertebrae defining an intervertebral foramen through which one of the dorsal root ganglia of FIG. 3B can extend outward from the spinal cord of FIG. 3B;

FIG. 4 is a schematic side view of one embodiment of components for a system, kit, or method for implanting a lead for stimulation of the dorsal root ganglion of a patient including an introducer, a guidewire, and a lead, according to the invention;

FIG. 5A is a schematic perspective view of the spinal cord of FIG. 3A disposed along a longitudinal transverse view of a portion of the vertebral column of FIG. 3B, where an introducer is used to advance a guidewire into the epidural space through an intervertebral foramen, according to the invention;

FIG. 5B is a schematic perspective view of the spinal cord of FIG. 3A disposed along a longitudinal transverse view of a portion of the vertebral column of FIG. 3B, where a lead is being advanced over the guidewire, according to the invention;

FIG. 5C is a schematic perspective view of the spinal cord of FIG. 3A disposed along a longitudinal transverse view of a portion of the vertebral column of FIG. 3B, where the lead is placed for stimulation of the dorsal root ganglion, according to the invention;

FIG. 6 is a schematic side view of one embodiment of a flat, blunt tip for the introducer of FIG. 4, according to the invention; and

FIG. 7 is a schematic overview of one embodiment of components of an electrical stimulation system, according to the invention.

### DETAILED DESCRIPTION

The present invention is directed to the area of implantable electrical stimulation systems and methods of making and using the systems. The present invention is also directed to electrical stimulation systems for stimulation of dorsal root ganglia, as well as methods of making and using the electrical stimulation systems.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, deep brain stimulation leads, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference.

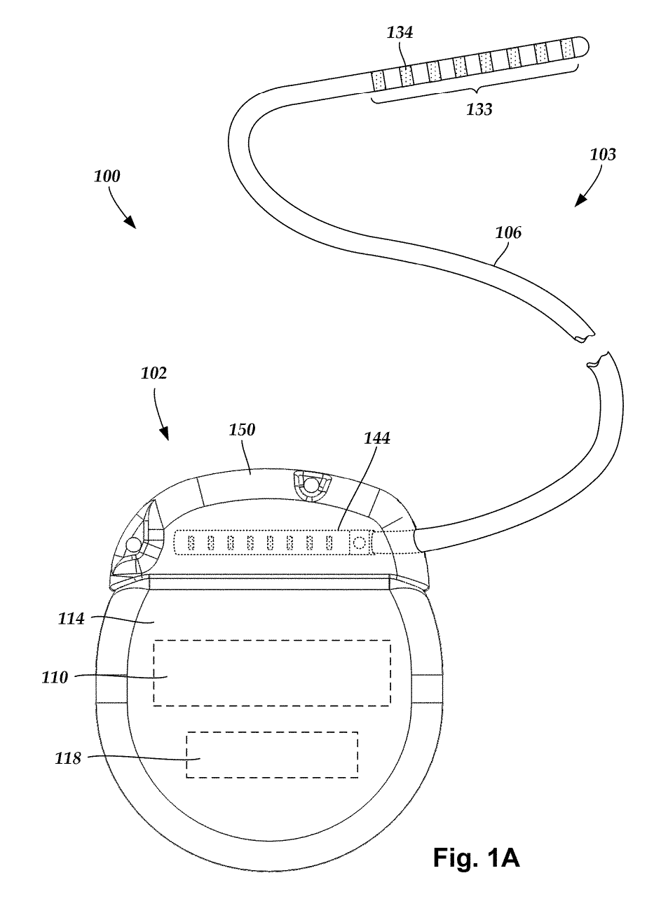

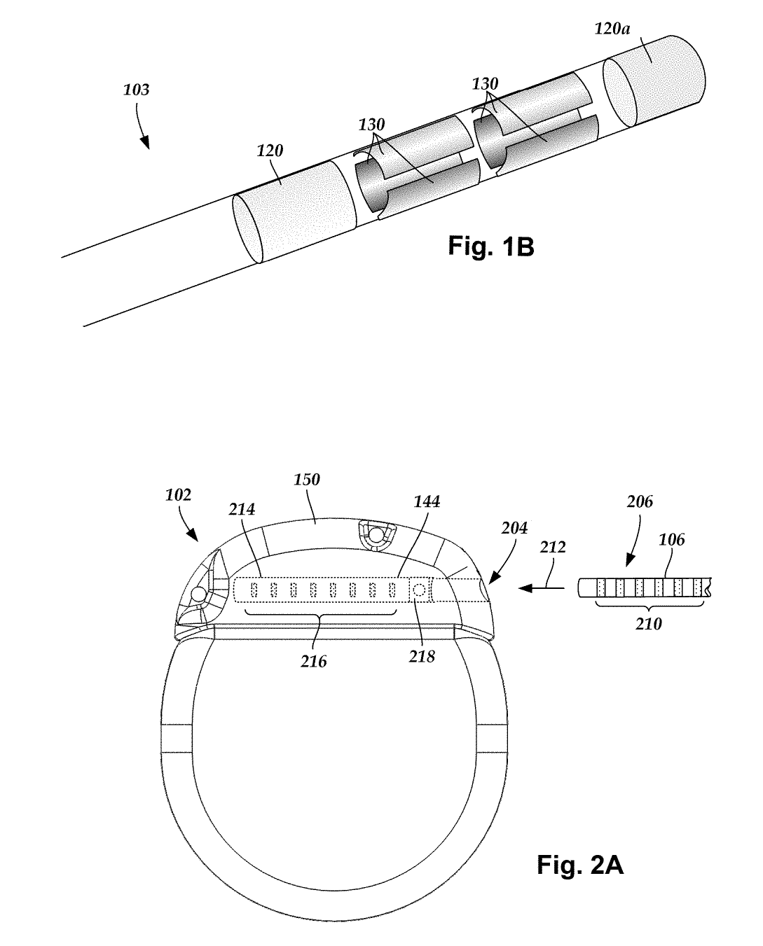

FIG. 1A illustrates schematically one embodiment of an electrical stimulation system 100. The electrical stimulation system 100 includes a control module (e.g., a stimulator or pulse generator) 102 and a percutaneous lead 103. The lead 103 includes a plurality of electrodes 134 that form an array of electrodes 133. The control module 102 typically includes an electronic subassembly 110 and an optional power source 118 disposed in a sealed housing 114. The lead 103 includes a lead body 106 coupling the control module 102 to the plurality of electrodes 134. In at least some embodiments, the lead body 106 is isodiametric.

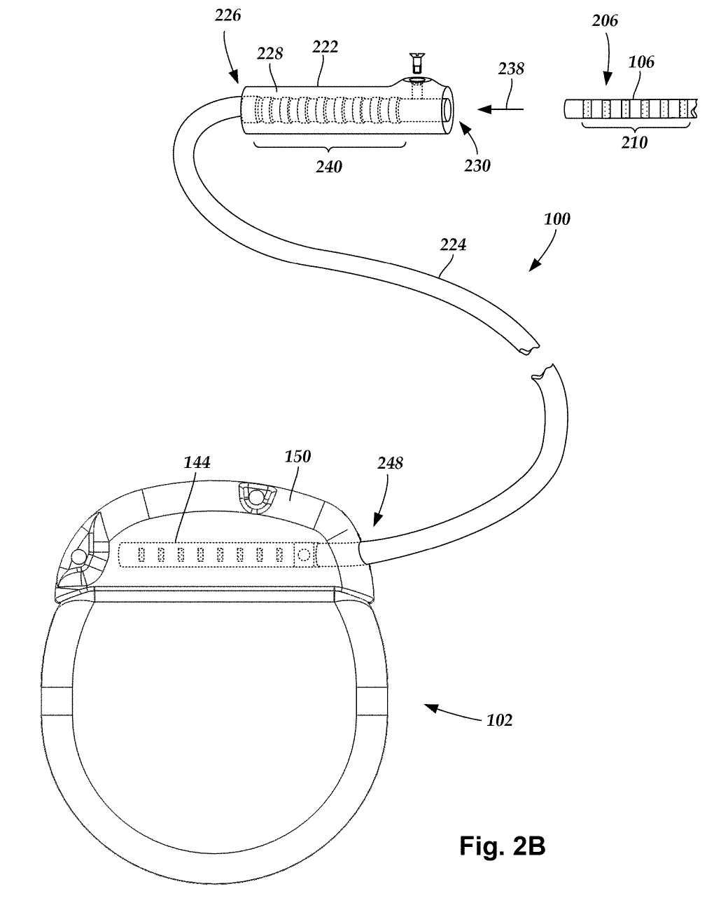

The control module 102 typically includes one or more connector assemblies 144 into which the proximal end of the lead body 106 can be plugged to make an electrical connection via connector contacts (e.g., 216 in FIG. 2A) disposed in the connector assembly 144 and terminals (e.g., 210 in FIG. 2A) disposed along the lead body 106. The connector contacts are coupled to the electronic subassembly 110 and the terminals are coupled to the electrodes 134. Optionally, the control module 102 may include a plurality of connector assemblies 144.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions (not shown) can be disposed between the lead body 106 and the control module 102 to extend the distance between the lead body 106 and the control module 102.

The electrical stimulation system or components of the electrical stimulation system, including the lead body 106 and the control module 102, are typically implanted into the body of a patient. The electrical stimulation system can be used for a variety of applications including, but not limited to, spinal cord stimulation, brain stimulation, neural stimulation, muscle activation via stimulation of nerves innervating muscle, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, or titanium.

The number of electrodes 134 in the array of electrodes 133 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. In FIG. 1A, eight electrodes 134 are shown. The electrodes 134 can be formed in any suitable shape including, for example, round, oval, triangular, rectangular, pentagonal, hexagonal, heptagonal, octagonal, or the like. In the illustrated leads, the electrodes are ring electrodes. Any number of ring electrodes can be disposed along the length of the lead body including, for example, one, two three, four, five, six, seven, eight, nine, ten, eleven, twelve, thirteen, fourteen, fifteen, sixteen or more ring electrodes. It will be understood that any number of ring electrodes can be disposed along the length of the lead body.

FIG. 1B illustrates a distal end of a lead 103 with a ring electrode 120, a tip electrode 120a, and six segmented electrodes 130. Segmented electrodes may provide for superior current steering than ring electrodes because target structures may not be disposed symmetrically about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to target tissue, while potentially avoiding stimulation of other tissue. Examples of leads with segmented electrodes include U.S. Patent Applications Publication Nos. 2010/0268298; 2011/0005069; 2011/0078900; 2011/0130803; 2011/0130816; 2011/0130817; 2011/0130818; 2011/0078900; 2011/0238129; 2011/0313500; 2012/0016378; 2012/0046710; 2012/0071949; 2012/0165911; 2012/197375; 2012/0203316; 2012/0203320; 2012/0203321; 2013/0197602; 2013/0261684; 2013/0325091; 2013/0317587; 2014/0039587; 2014/0353001; 2014/0358209; 2014/0358210; 2015/0018915; 2015/0021817; 2015/0045864; 2015/0021817; 2015/0066120; 2013/0197424; 2015/0151113; 2014/0358207; and U.S. Pat. No. 8,483,237, all of which are incorporated herein by reference in their entireties. Examples of leads with tip electrodes include at least some of the previously cited references, as well as U.S. Patent Applications Publication Nos. 2014/0296953 and 2014/0343647, all of which are incorporated herein by reference in their entireties. A lead with segmented electrodes may be a directional lead that can provide stimulation in a particular direction using the segmented electrodes.

Any number of segmented electrodes 130 may be disposed on the lead body including, for example, one, two three, four, five, six, seven, eight, nine, ten, eleven, twelve, thirteen, fourteen, fifteen, sixteen or more segmented electrodes 130. It will be understood that any number of segmented electrodes 130 may be disposed along the length of the lead body. A segmented electrode 130 typically extends only 75%, 67%, 60%, 50%, 40%, 33%, 25%, 20%, 17%, 15%, or less around the circumference of the lead. The segmented electrodes 130 may be grouped into sets of segmented electrodes, where each set is disposed around a circumference of the lead 103 at a particular longitudinal portion of the lead 103. The lead 102 may have any number segmented electrodes 130 in a given set of segmented electrodes. The lead 103 may have one, two, three, four, five, six, seven, eight, or more segmented electrodes 130 in a given set. The segmented electrodes 130 may vary in size and shape. In some embodiments, the segmented electrodes 130 are all of the same size, shape, diameter, width or area or any combination thereof. In some embodiments, the segmented electrodes 130 of each circumferential set (or even all segmented electrodes disposed on the lead 103) may be identical in size and shape.

Each set of segmented electrodes 130 may be disposed around the circumference of the lead body to form a substantially cylindrical shape around the lead body. The spacing between individual electrodes of a given set of the segmented electrodes may be the same, or different from, the spacing between individual electrodes of another set of segmented electrodes on the lead 103. In at least some embodiments, equal spaces, gaps or cutouts are disposed between each segmented electrode 130 around the circumference of the lead body. In other embodiments, the spaces, gaps or cutouts between the segmented electrodes 130 may differ in size or shape. In other embodiments, the spaces, gaps, or cutouts between segmented electrodes 130 may be uniform for a particular set of the segmented electrodes 130, or for all sets of the segmented electrodes 130. The sets of segmented electrodes 130 may be positioned in irregular or regular intervals along a length the lead body.

The electrodes of the lead body 106 are typically disposed in, or separated by, a non-conductive, biocompatible material including, for example, silicone, polyurethane, and the like or combinations thereof. The lead body 106 may be formed in the desired shape by any process including, for example, extruding, molding (including injection molding), casting, and the like. Electrodes and connecting wires can be disposed onto or within a lead body either prior to or subsequent to a molding or casting process. The non-conductive material typically extends from the distal end of the lead body 106 to the proximal end of the lead body 106.

Terminals (e.g., 210 in FIG. 2A) are typically disposed at the proximal end of the lead body 106 for connection to corresponding conductive contacts (e.g., 216 in FIG. 2A) in one or more connector assemblies (e.g., 144 in FIG. 1A) disposed on, for example, the control module 102 (or to other devices, such as conductive contacts on a lead extension, an operating room cable, a splitter, an adaptor, or the like).

Conductive wires extend from the plurality of terminals (see e.g., 210 in FIG. 2A) to the plurality of electrodes 133. Typically, each of the plurality of terminals is electrically coupled to at least one of the plurality of electrodes 133. In some embodiments, each of the plurality of terminals is coupled to a single electrode 134 of the plurality of electrodes 133.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the lead 103. The one or more lumens may, optionally, be flushed continually, or on a regular basis, with saline or the like. The one or more lumens can be permanently or removably sealable at the distal end.

As discussed above, the lead body 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1A, the lead body 106 includes eight terminals that are shown coupled with eight conductive contacts disposed in the connector assembly 144.

FIG. 2A is a schematic side view of one embodiment of a connector assembly 144 disposed on the control module 102. In FIG. 2A, the proximal end 206 of the lead body 106 is shown configured and arranged for insertion to the control module 102.

In FIG. 2A, the connector assembly 144 is disposed in the header 150. In at least some embodiments, the header 150 defines a port 204 into which the proximal end 206 of the lead body 106 with terminals 210 can be inserted, as shown by directional arrows 212, in order to gain access to the connector contacts disposed in the connector assembly 144.

The connector assembly 144 includes a connector housing 214 and a plurality of connector contacts 216 disposed therein. Typically, the connector housing 214 defines a port (not shown) that provides access to the plurality of connector contacts 216. In at least some embodiments, the connector assembly 144 further includes a retaining element 218 configured and arranged to fasten the corresponding lead body 106 or lead retention sleeve to the connector assembly 144 when the lead body 106 is inserted into the connector assembly 144 to prevent undesired detachment of the lead body 106 from the connector assembly 144. For example, the retaining element 218 may include an aperture 220 through which a fastener (e.g., a set screw, pin, or the like) may be inserted and secured against an inserted lead body 106 or lead retention sleeve.

When the lead body 106 is inserted into the port 204, the connector contacts 216 can be aligned with the terminals 210 disposed on the lead body 106 to electrically couple the control module 102 to the electrodes (134 of FIG. 1A) disposed at a distal end of the lead body 106. Examples of connector assemblies in control modules are found in, for example, U.S. Pat. No. 7,244,150 and U.S. Patent Application Publication No. 2008/0071320, which are incorporated by reference.

In at least some embodiments, the electrical stimulation system includes one or more lead extensions. The lead body 106 can be coupled to one or more lead extensions which, in turn, are coupled to the control module 102. In FIG. 2B, a lead extension connector assembly 222 is disposed on a lead extension 224. The lead extension connector assembly 222 is shown disposed at a distal end 226 of the lead extension 224. The lead extension connector assembly 222 includes a contact housing 228. The contact housing 228 defines at least one port 230 into which a proximal end 206 of the lead body 106 with terminals 210 can be inserted, as shown by directional arrow 238. The lead extension connector assembly 222 also includes a plurality of connector contacts 240. When the lead body 106 is inserted into the port 230, the connector contacts 240 disposed in the contact housing 228 can be aligned with the terminals 210 on the lead body 106 to electrically couple the lead extension 224 to the electrodes (134 of FIG. 1A) disposed at a distal end (not shown) of the lead body 106.

The proximal end of a lead extension can be similarly configured and arranged as a proximal end of a lead body. The lead extension 224 may include a plurality of conductive wires (not shown) that electrically couple the connector contacts 240 to terminal on a proximal end 248 of the lead extension 224. The conductive wires disposed in the lead extension 224 can be electrically coupled to a plurality of terminals (not shown) disposed on the proximal end 248 of the lead extension 224. In at least some embodiments, the proximal end 248 of the lead extension 224 is configured and arranged for insertion into a lead extension connector assembly disposed in another lead extension. In other embodiments (as shown in FIG. 2B), the proximal end 248 of the lead extension 224 is configured and arranged for insertion into the connector assembly 144 disposed on the control module 102.

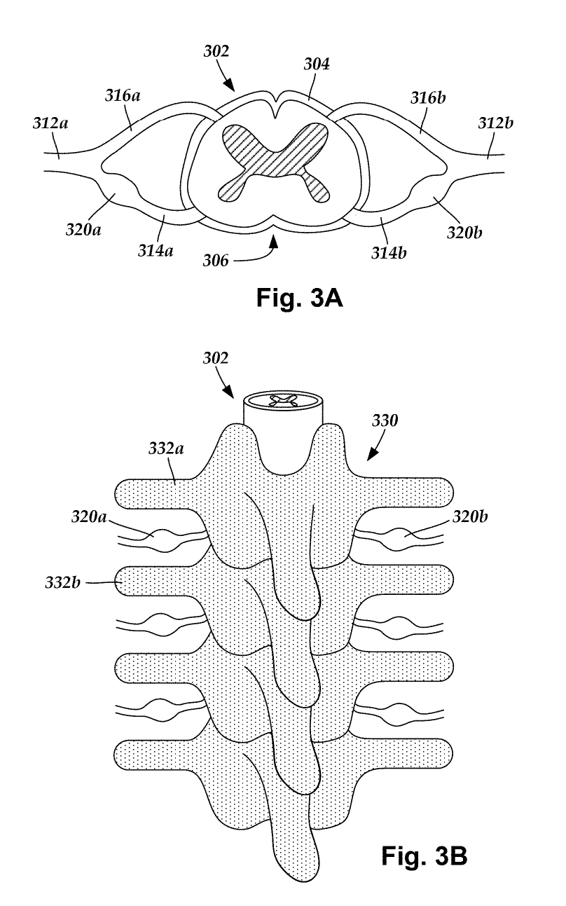

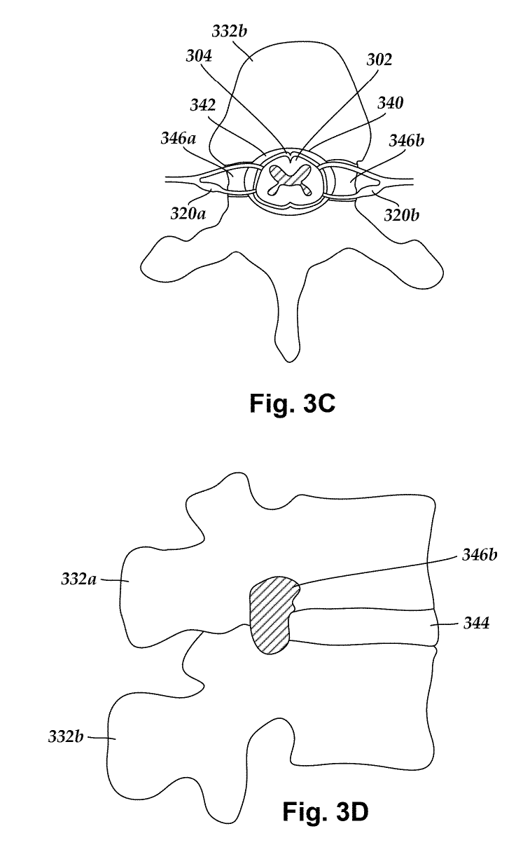

Turning to FIG. 3A, in at least some embodiments one or more dorsal root ganglia (“DRG”) are potential target stimulation locations. FIG. 3A schematically illustrates a transverse cross-sectional view of a spinal cord 302 surrounded by dura 304. The spinal cord 302 includes a midline 306 and a plurality of levels from which spinal nerves 312a and 312b extend. In at least some spinal cord levels, the spinal nerves 312a and 312b extend bilaterally from the midline 306 of the spinal cord 302. In FIG. 3A, the spinal nerves 312a and 312b are shown attaching to the spinal cord 302 at a particular spinal cord level via corresponding dorsal roots 314a and 314b and corresponding ventral (or anterior) roots 316a and 316b. Typically, the dorsal roots 314a and 314b relay sensory information into the spinal cord 302 and the ventral roots 316a and 316b relay motor information outward from the spinal cord 302. The DRG 320a and 320b are nodules of cell bodies that are disposed along the dorsal roots 316a and 316b in proximity to the spinal cord 302.

FIG. 3B schematically illustrates a perspective view of a portion of the spinal cord 302 disposed along a portion of a vertebral column 330. The vertebral column 330 includes stacked vertebrae, such as vertebrae 332a and 332b, and a plurality of DRGs 320a and 320b extending outwardly bilaterally from the spinal cord 302 at different spinal cord levels.

FIG. 3C schematically illustrates a top view of a portion of the spinal cord 302 and surrounding dura 304 disposed in a vertebral foramen 340 defined in the vertebra 332b. The vertebrae, such as the vertebrae 332a and 332b, are stacked together and the vertebral foramina 340 of the vertebrae collectively form a spinal canal through which the spinal cord 302 extends. The space within the spinal canal between the dura 304 and the walls of the vertebral foramen 340 defines the epidural space 342. Intervertebral foramina 346a and 346b, defined bilaterally along sides of the vertebra 332b, form openings through the vertebra 332b between the epidural space 342 and the environment external to the vertebra 332b.

FIG. 3D schematically illustrates a side view of two vertebrae 332a and 332b coupled to one another by a disc 344. In FIG. 3D, the intervertebral foramen 346b is shown defined between the vertebrae 332a and 332b. The intervertebral foramen 346b provides an opening for one or more of the dorsal root 314b, ventral root 316b, and DRG 320b to extend outwardly from the spinal cord 302 to the environment external to the vertebrae 332a and 332b.

There can be challenges to implanting a lead for stimulation of a dorsal root ganglion (DRG). For example, in at least some embodiments, the angle of insertion of the lead into the patient may be critical and can be different than that used for traditional spinal cord stimulation. This angle can vary significantly depending on the entry location and desired stimulation site. Moreover, the angle and other aspects of the implantation may vary depending on the stimulation target and the spinal cord level for the stimulation.

In addition, to better visualize where the lead is in the epidural space with respect to the foramen, both A/P (anterior/posterior) and lateral images are useful. However, taking multiple fluoroscopic images can be time consuming especially as the clinician is advancing a lead in the epidural space. Moreover, if imaging indicates the lead is not placed in the desired position, repositioning of the lead can take 30 minutes or more because of the challenges with placing the lead into the foramen.

Furthermore, many patients needing DRG stimulation have a lot of scar tissue in the epidural space, often due to failed back surgery. This may make advancing a lead in the epidural space and into the foramen difficult. Scar tissue may also cause kinking of the introducer or lead.

To address these challenges, a thin guidewire with an electrode can first be inserted into the epidural space and through the foramen to the vicinity of the dorsal root ganglion. The electrode on the guidewire can be used for mapping a region around the dorsal root ganglion and for identifying a desired stimulation site. Because the guidewire is smaller in diameter than the lead, a thinner introducer (e.g., a needle) can be used for implantation. The thinner needle and guidewire can often penetrate scar tissue easier than a larger lead and its introducer. This can also reduce the likelihood of kinking. Moreover, repositioning the guidewire (for example, for mapping or for identifying a suitable lead implantation site) may be easier due to its smaller diameter. Repositioning of the lead may also be unnecessary due the mapping of the region around the dorsal root ganglion. Once the desired lead implantation site is identified, the lead can be inserted into the patient. In at least some embodiments, the lead is inserted over the guidewire to direct the lead to the desired implantation site.

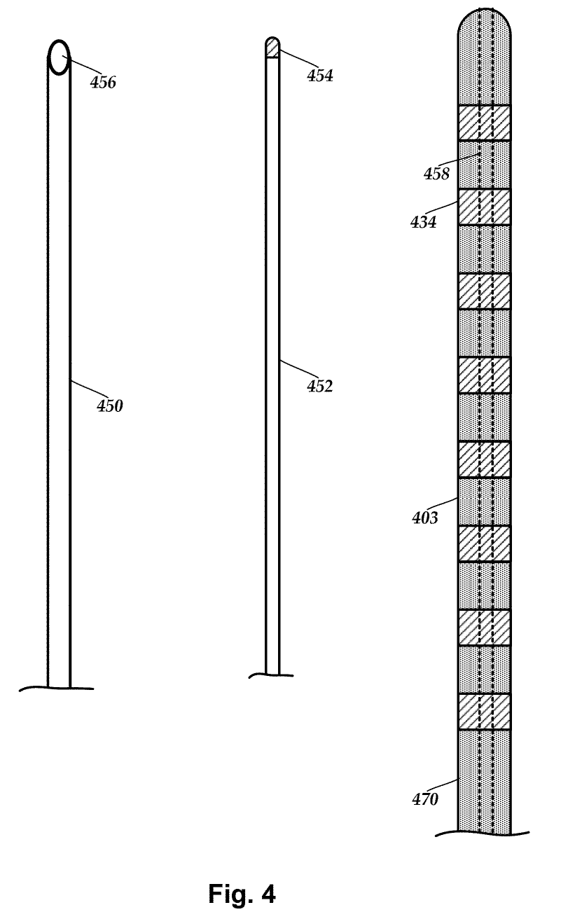

FIG. 4 illustrates one embodiment of components that can form or be part of a system, kit, or method for electrical stimulation of a dorsal root ganglion. These components include an introducer 450, a guidewire 452, and an electrical stimulation lead 403. In at least some embodiments, the guidewire 452 includes an electrode 454 disposed on, or near, a distal end of guidewire. A conductor (not shown) will extend along the guidewire 452 from the electrode 454 to a proximal end of the guidewire so that the guidewire can be coupled to a device for providing or receiving electrical signals from the electrode 454. Although a single electrode 454 is illustrated, in some embodiments the guidewire 452 includes two or more electrodes which may be electrically coupled together or may be independent of each other with separate conductors extending along the guidewire.

The introducer 450 defines a lumen 456 through which the guidewire 452 can be delivered. The electrical stimulation lead 403 includes a central lumen 458, sized to receive the guidewire 452, so that the lead can be inserted into the patient over the guidewire.

The electrical stimulation lead 403 includes a lead body 470 and electrodes 434. As examples, any of the leads described herein can be used as lead 403.

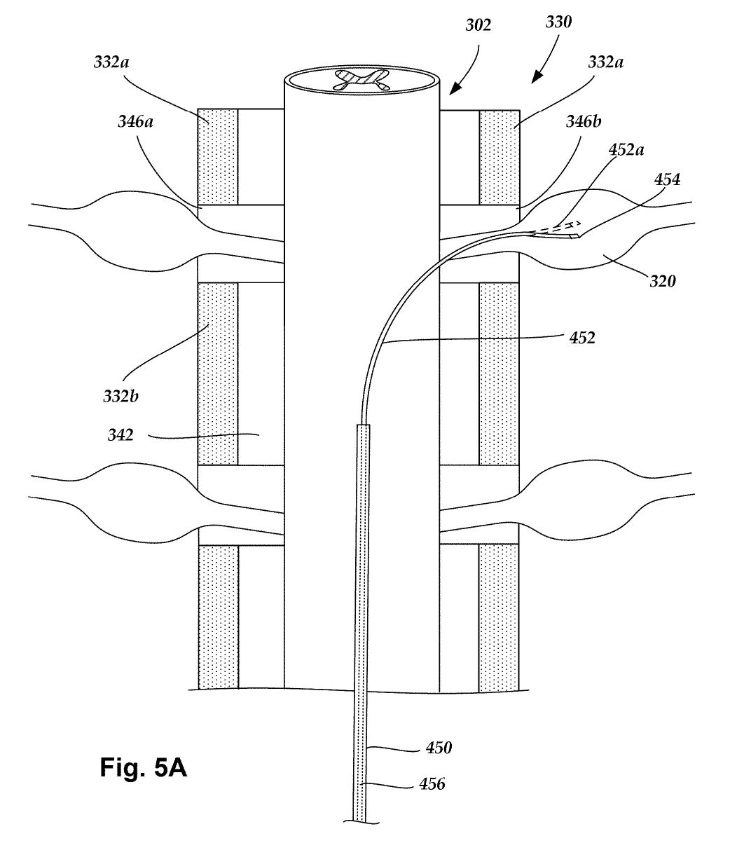

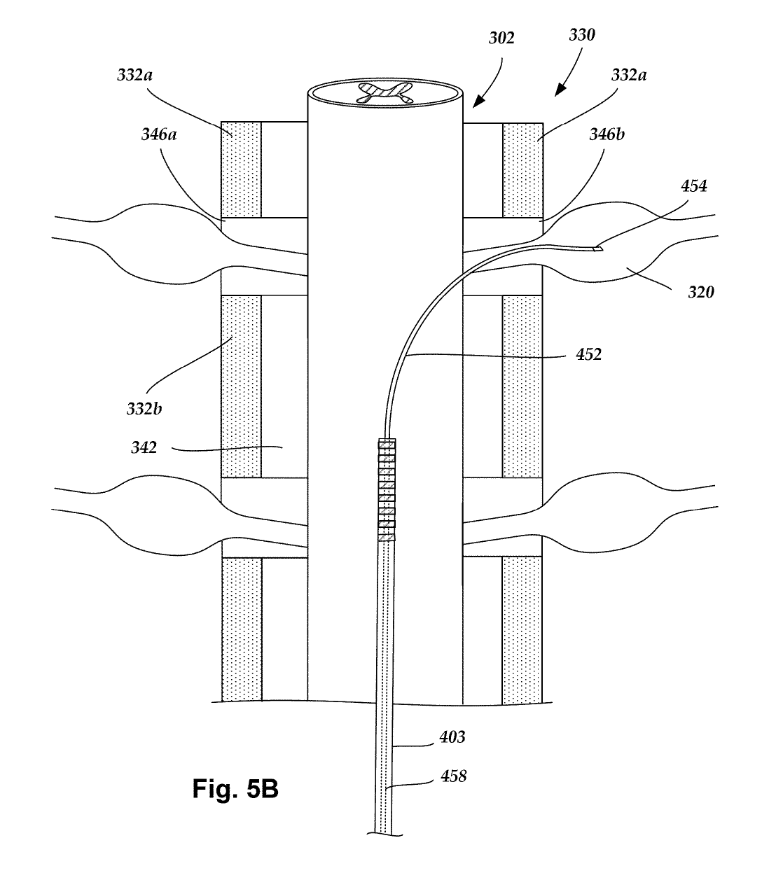

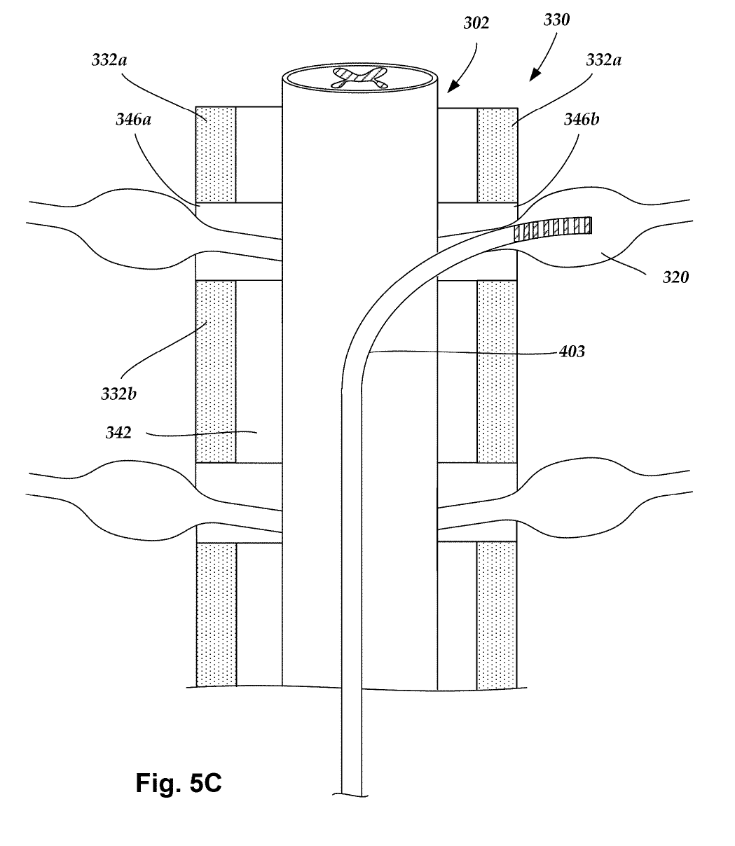

Turning to FIG. 5A, the guidewire 452 can be inserted into a patient using the introducer 450 in order to identify a target stimulation location related to the patient's DRG. In the illustrated example, the guidewire is introduced through the patient's epidural space. Although the DRG are not within the epidural space, one or more of the DRG may be accessible to the guidewire 452 from within the epidural space via the intervertebral foramina.

FIGS. 5A-5C are schematic perspective views of the spinal cord 302 disposed along a longitudinal transverse view of a portion of the vertebral column 330. The portion of the vertebral column 330 shown in FIGS. 5A-5C includes the vertebrae 332a and 332b and intervertebral foramina 346a and 346b defined between the vertebrae 332a and 332b on opposing sides of the vertebral column 330. A DRG 320 extends outward from one side of the spinal cord 302 and through the intervertebral foramen 346b.

The guidewire 452 can be advanced out of the epidural space through one of the intervertebral foramen, and for placement near, adjacent, in contact with, or inserted into the desired DRG 320. In at least some embodiments, the introducer 450 can also penetrate and extend through the intervertebral foramen 346a during delivery and placement of the guidewire 452. In other embodiments, the introducer 450 may only enter the epidural space and the guidewire 452 is pushed through the intervertebral foramen 346a. Once the guidewire 452 is placed, the introducer 450 can be removed or backed off, as illustrated in FIG. 5A.

While a conventional introducer used for implanting a stimulation lead is often 14 gauge (0.083″ or 0.21 cm nominal outer diameter) or larger, a smaller introducer 450 of, for example, 20 gauge (0.036″ or 0.091 cm nominal outer diameter) or smaller can be used for placement of the mapping guidewire 452. Using a smaller introducer 450 for placement of the guidewire 452 can provide for more fine adjustments of guidewire position and may facilitate more precise locating of a point of entry into the epidural space or through the foramen. Additionally, and especially in the cervical region, a smaller introducer provides lower risk of dura puncture.

Advancing a small mapping guidewire 452 into and through the foramen 346b and moving the small mapping guidewire with respect to the DRG 320 may be easier and less time consuming than using a lead to map the DRG. Furthermore, this guidewire can be steerable to allow for manipulation within the epidural space, through the foramen, and into a desired location on or near the DRG. Repositioning of the introducer 450 or guidewire 452 is easier and faster with a smaller introducer instead of the conventional larger lead and its introducer. For example, FIG. 5, illustrates in dotted lines 452a, a new position for the distal end of the guidewire 452 as the guidewire is steered or repositioned relative to the DRG.

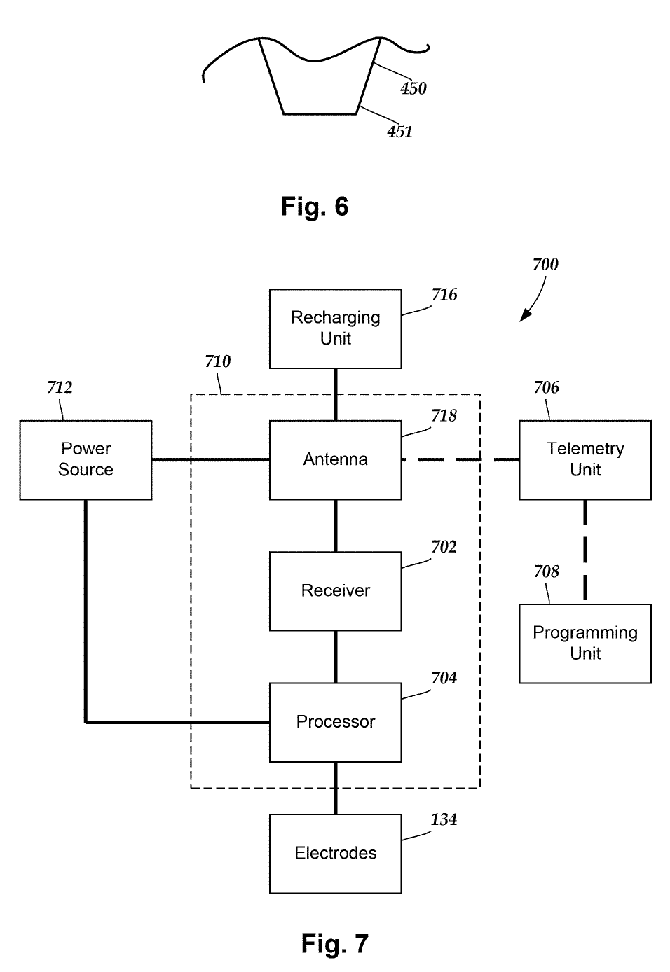

In addition, advancing a small introducer 450 into the epidural space and through scar tissue will typically be easier than with a conventional lead. In at least some embodiments, the guidewire 452 or introducer 450 is used to create an initial path through the scar tissue that can then be traversed with a larger diameter object such as a lead or a tool specifically designed to clear the scar tissue obstructions. In at least some embodiments, the introducer 450 can have a flat, blunt tip 451, as illustrated in FIG. 6, rather than a conventional rounded tip, to facilitate penetration of scar tissue. Additionally or alternatively, the guidewire 452 can have the blunt tip. Additionally, the introducer 340 may have a reinforced mesh configuration to reduce kinking.

The guidewire 452 and its associated electrode 454 can be used to map or otherwise test the response of the patient tissue to electrical stimulation. Additionally or alternatively, the guidewire 452 and its associated electrode 454 can be used to map the electrical signals from patient tissue. In particular, the electrode 454 of the guidewire 452 can be used to map the space in and around the DRG. The mapping can be used to find a desirable location for lead placement.

FIG. 5B illustrates the insertion of the lead 403 over the guidewire 452. During the insertion process, the introducer 450 is withdrawn leaving the guidewire 452 which is then fed into the central lumen 458 of the lead 403 and the lead is then pushed along the guidewire. It will be understood that in other embodiments, the guidewire 452 can be withdrawn and the lead 403 can be implanted using a lead introducer (not shown) to for placement of the distal portion of the lead at the implantation site identified using the guidewire.

FIG. 5C illustrates the lead 403 placed at the desired stimulation site. The guidewire 452 may remain implanted or may be withdrawn following placement of the lead. In at least some embodiments, the lead 403 is anchored to patient tissue using a lead anchor, such as, for example, the Clik™ anchor (Boston Scientific Corporation.)

FIG. 7 is a schematic overview of one embodiment of components of an electrical stimulation system 700 including an electronic subassembly 710 disposed within a control module. It will be understood that the electrical stimulation system can include more, fewer, or different components and can have a variety of different configurations including those configurations disclosed in the stimulator references cited herein.

Some of the components (for example, power source 712, antenna 718, receiver 702, and processor 704) of the electrical stimulation system can be positioned on one or more circuit boards or similar carriers within a sealed housing of an implantable pulse generator, if desired. Any power source 712 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 718 or a secondary antenna. The external power source can be in a device that is mounted on the skin of the user or in a unit that is provided near the user on a permanent or periodic basis.

If the power source 712 is a rechargeable battery, the battery may be recharged using the optional antenna 718, if desired. Power can be provided to the battery for recharging by inductively coupling the battery through the antenna to a recharging unit 716 external to the user. Examples of such arrangements can be found in the references identified above.

In one embodiment, electrical current is emitted by the electrodes 134 on the lead body to stimulate nerve fibers, muscle fibers, or other body tissues near the electrical stimulation system. A processor 704 is generally included to control the timing and electrical characteristics of the electrical stimulation system. For example, the processor 704 can, if desired, control one or more of the timing, frequency, amplitude, width, and waveform of the pulses. In addition, the processor 704 can select which electrodes can be used to provide stimulation, if desired. In some embodiments, the processor 704 may select which electrode(s) are cathodes and which electrode(s) are anodes. In some embodiments, the processor 704 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 708 that, for example, allows modification of pulse characteristics. In the illustrated embodiment, the processor 704 is coupled to a receiver 702 which, in turn, is coupled to the optional antenna 718. This allows the processor 704 to receive instructions from an external source to, for example, direct the pulse characteristics and the selection of electrodes, if desired.

In one embodiment, the antenna 718 is capable of receiving signals (e.g., RF signals) from an external telemetry unit 706 which is programmed by a programming unit 708. The programming unit 708 can be external to, or part of, the telemetry unit 706. The telemetry unit 706 can be a device that is worn on the skin of the user or can be carried by the user and can have a form similar to a pager, cellular phone, or remote control, if desired. As another alternative, the telemetry unit 706 may not be worn or carried by the user but may only be available at a home station or at a clinician's office. The programming unit 708 can be any unit that can provide information to the telemetry unit 706 for transmission to the electrical stimulation system 700. The programming unit 708 can be part of the telemetry unit 706 or can provide signals or information to the telemetry unit 706 via a wireless or wired connection. One example of a suitable programming unit is a computer operated by the user or clinician to send signals to the telemetry unit 706.

The signals sent to the processor 704 via the antenna 718 and receiver 702 can be used to modify or otherwise direct the operation of the electrical stimulation system.

For example, the signals may be used to modify the pulses of the electrical stimulation system such as modifying one or more of pulse width, pulse frequency, pulse waveform, and pulse amplitude. The signals may also direct the electrical stimulation system 700 to cease operation, to start operation, to start charging the battery, or to stop charging the battery. In other embodiments, the stimulation system does not include an antenna 718 or receiver 702 and the processor 704 operates as programmed.

Optionally, the electrical stimulation system 700 may include a transmitter (not shown) coupled to the processor 704 and the antenna 718 for transmitting signals back to the telemetry unit 706 or another unit capable of receiving the signals. For example, the electrical stimulation system 700 may transmit signals indicating whether the electrical stimulation system 700 is operating properly or not or indicating when the battery needs to be charged or the level of charge remaining in the battery. The processor 704 may also be capable of transmitting information about the pulse characteristics so that a user or clinician can determine or verify the characteristics.

The above specification and examples provide a description of the arrangement and use of the invention. Since many embodiments of the invention can be made without departing from the spirit and scope of the invention, the invention also resides in the claims hereinafter appended.

What is claimed as new and desired to be protected by Letters Patent of the United States is:

1. A method for implanting a lead for stimulation of a dorsal root ganglion of a patient, the method comprising:
advancing a distal portion of a guidewire using an introducer into an epidural space of the patient and through a foramen of the patient to a position near the dorsal root ganglion, the guidewire comprising an electrode in the distal portion of the guidewire;
mapping a region around the dorsal root ganglion using the electrode of the guidewire to identify a lead implantation site;
removing the introducer; and
advancing the lead over the guidewire, with a portion of the guidewire disposed in a lumen of the lead, to position a distal portion of the lead at the lead implantation site.

2. The method of claim 1, wherein advancing the distal portion of the guidewire comprises advancing the introducer and the distal portion of the guidewire through the foramen of the patient.

3. The method of claim 1, wherein the introducer has a flat, blunt tip to facilitate penetration of scar tissue around the foramen.

4. The method of claim 1, wherein mapping the region around the dorsal root ganglion comprises stimulation of patient tissue using the electrode of the guidewire.

5. The method of claim 1, wherein mapping the region around the dorsal root ganglion comprises receiving electrical signals from patient tissue using the electrode of the guidewire.

6. The method of claim 1, wherein the introducer is no more than 20 gauge.

7. The method of claim 1, further comprising repositioning the distal portion of the guidewire to another site relative to the dorsal root ganglion.

8. The method of claim 1, wherein the introducer comprises a reinforced mesh to reduce kinking.

9. A method for implanting a lead for stimulation of a dorsal root ganglion of a patient, the method comprising:
advancing a distal portion of a guidewire through an epidural space of the patient and through a foramen of the patient to a position near the dorsal root ganglion, the guidewire comprising an electrode in the distal portion of the guidewire;
mapping a portion of the patient tissue adjacent the distal portion of the guidewire using the electrode;
repositioning the distal portion of the guidewire to a lead implantation site relative to the dorsal root ganglion and mapping an additional portion of the patient tissue using the electrode; and
advancing the lead over the guidewire, with a portion of the guidewire disposed in a lumen of the lead, to position a distal portion of the lead at the lead implantation site.

10. The method of claim 9, wherein advancing the distal portion of the guidewire comprises advancing the guidewire through an introducer.

11. The method of claim 10, wherein advancing the distal portion of the guidewire further comprises advancing the introducer and the distal portion of the guidewire through the foramen of the patient.

12. The method of claim 9, wherein the introducer has a flat, blunt tip to facilitate penetration of scar tissue around the foramen.

13. The method of claim 9, wherein the introducer is no more than 20 gauge.

14. The method of claim 9, wherein mapping the portion of the patient tissue comprises stimulating patient tissue using the electrode of the guidewire.

15. The method of claim 9, wherein mapping the portion of the patient tissue comprises receiving electrical signals from patient tissue using the electrode of the guidewire.

16. The method of claim 9, wherein the introducer comprises a reinforced mesh to reduce kinking.

17. A kit for implanting a lead for stimulation of a dorsal root ganglion of a patient, the kit comprising:
a guidewire with an electrode disposed at a distal end of the guidewire;
an introducer having a lumen for receiving the guidewire; and
a lead comprising a lead body and a plurality of electrodes disposed along a distal end of the lead body, the lead body defining a central lumen for receiving the guidewire.

18. The kit of claim 17, wherein the introducer has a blunt tip for penetrating scar tissue.

19. The kit of claim 17, wherein the introducer is no more than 20 gauge.

20. The kit of claim 17, wherein the introducer comprises a reinforced mesh to reduce kinking.
